(19) United States
(12) Patent Application Publication
(10) Pub. No.: US 2021/0118546 A1
(43) Pub. Date: Apr. 22, 2021

(54) EMOTION DETECTION FROM CONTEXTUAL SIGNALS FOR SURFACING WELLNESS INSIGHTS

(71) Applicant: Microsoft Technology Licensing, LLC, Redmond, WA (US)

(72) Inventor: Subramanian Ramakrishnan, Karnataka (IN)

(21) Appl. No.: 16/659,885

(22) Filed: Oct. 22, 2019

(51) Int. Cl. G16H 20/70; G16H 50/20; G16H 50/30; G06F 17/27

(52) U.S. Cl. CPC G16H 20/70; G06F 17/2785; G16H 50/30; G16H 50/20

(57) ABSTRACT

In non-limiting examples of the present disclosure, systems, methods and devices for surfacing wellness recommendations are presented. A plurality of signals related to a user may be received. The plurality of signals may comprise: an active duration of time spent composing or reviewing an email and a biometric signal associated with the user. The biometric signal may comprise at least one of: a blood pressure value for the user during a time that the email was being composed or reviewed, and a heartrate value during a time that the email was being composed or reviewed. An anxiety score associated with the email may be generated for the user. A determination may be made that the anxiety score is above a threshold baseline value for the user. A wellness recommendation related to the email may be caused to be surfaced.

### BACKGROUND

It has become common for enterprises to make substantial investments in their employees' health and wellbeing. Enterprises understand that such investments are worthwhile because they are better able to retain talent. Additionally, the work product from healthy and happy employees is generally better. In an enterprise workplace, emails are a primary mode of communication, and a great deal of employee time is spent interacting with email clients. Email-related anxiety is a pressing problem that impacts employee productivity and health.

It is with respect to this general technical environment that aspects of the present technology disclosed herein have been contemplated. Furthermore, although a general environment has been discussed, it should be understood that the examples described herein should not be limited to the general environment identified in the background.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description or may be learned by practice of the disclosure.

Non-limiting examples of the present disclosure describe systems, methods and devices for generating and surfacing wellness recommendations. A user may provide a wellness insight service with access to data from one or more computing devices and/or applications associated with a user account (e.g., a user ID and password). The signals may relate to work events that the user partakes in. The work events may include composing emails, reviewing emails, and attending meetings, for example. The wellness insight service may receive data from email applications, electronic calendar applications, contacts applications, task completion applications, word processing applications, etc. The wellness insight service may also receive data from hardware associated with a user's computing devices (e.g., camera data, audio data). In some examples, the wellness insight service may be granted with access to data associated with a user's secondary devices (e.g., smartwatches, fitness trackers). The secondary devices may provide the wellness insight service with biometric data (e.g., heartrate data, blood pressure data, etc.).

The wellness insight service may analyze received data from times corresponding to work events and generate an anxiety score for a user for those events. If the anxiety score is above a threshold baseline value, the wellness insight service may cause one or more wellness recommendations to be surfaced. The wellness recommendations may include a description of the signals that an anxiety score was generated from. The wellness recommendations may additionally or alternatively include suggestions for enhancing the user's wellness in relation to the anxiety.

### BRIEF DESCRIPTION OF THE DRAWINGS

Non-limiting and non-exhaustive examples are described with reference to the following figures:

FIG. 1 is a schematic diagram illustrating an example distributed computing environment for providing wellness insights in relation to a user's email.

FIG. 2 illustrates a distributed computing environment for generating and surfacing wellness insights in relation to user tasks.

FIG. 3 illustrates a computing environment, including a dashboard user interface, for surfacing wellness insights.

FIG. 4 illustrates a computing environment for surfacing wellness recommendations related to users' task and health signals.

FIG. 5 illustrates a computing environment for surfacing wellness insights in relation to an email that has been received.

FIG. 6 illustrates a computing environment for surfacing wellness insights in relation to a meeting.

FIG. 7 illustrates a computing environment for surfacing wellness insights in relation to a calendar application and health signals from a wearable device.

FIG. 8 is an exemplary method for surfacing wellness insights related to an outgoing email.

FIG. 9 is an exemplary method for surfacing wellness insights related to a received email.

FIGS. 10 and 11 are simplified diagrams of a mobile computing device with which aspects of the disclosure may be practiced.

FIG. 12 is a block diagram illustrating example physical components of a computing device with which aspects of the disclosure may be practiced.

FIG. 13 is a simplified block diagram of a distributed computing system in which aspects of the present disclosure may be practiced.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Non-limiting examples of the present disclosure describe systems, methods and devices for generating and surfacing wellness insights. A user account (e.g., user ID and password) may be associated with a wellness insight service. The wellness insight service may be executed on one or more client devices, on one or more server computing devices (i.e., in the cloud), and/or partially on one or more client devices and partially on one or more server computing devices. The wellness insight service may have been granted with access to data associated with the user account. For example, the user associated with the user account may have granted, via privacy settings, the wellness insight service with access to data from one or more productivity applications (e.g., email applications, word processing applications, presentation applications, spreadsheet applications, task completion applications, calendar applications, contacts applications, etc.) and/or client computing devices that execute/access those productivity applications. In some examples, the applications may be locally installed one or more client computing devices. In other examples, the applications may be web applications.

Examples of data (also described herein as “signals”) that the wellness insight service may be granted with access to from productivity applications and/or computing devices associated with the user account include: active duration of time spent on email authoring; active duration of time spent on email viewing/reading; number of times an email application/client has been manually refreshed; number of scrolls/highlights made to received emails; number of emails composed and/or sent; number of emails opened and/or read; recipient list for email; number of re-writes or corrections made during authoring of email; complexity of email (e.g., length, content type, etc.); volume of email sent on a day and/or during a temporal window; number of calendar meetings on a day and/or during a temporal window; audio data (recorded by a microphone); image data (captured by a camera); and/or rigor of key press on keyboard data (captured via haptic feedback sensors).

The user may have also granted the wellness insight service with access to data from one or more secondary devices (e.g., smartwatch data, fitness tracker data, digital assistant data, etc.). Examples of data (also described herein as “signals”) that the wellness insight service may be granted with access to from secondary devices include: heartrate values, blood pressure values, geo-location data, audio data, and image data.

Based on analyzing one or more of the above-described signals, the wellness insight service may identify a work event associated with a user, determine an anxiety level associated with the work event, and cause a wellness insight related to the work event to be surfaced if the anxiety level is determined to be above a threshold value. Work events may include email drafting events, email reading events, and meeting events, for example. The wellness insights that are surfaced may include a description of why the wellness insight service has determined that a user was anxious during a work event (e.g., a description of one or more signals that indicate a heightened anxiety level). For example, if the wellness insight service receives data from an email application indicating that a user spent above a threshold duration of time reading an email from a manager, and data indicating that the user's blood pressure and heartrate values were elevated above threshold values during that time, the wellness insight service may surface one or more insights describing that information. The wellness insights that are surfaced may additionally or alternatively include recommendations for increasing a user's wellness in relation to work events for which the wellness insight service has determined that a user's anxiety level is above a threshold value.

In some examples, wellness insights may be caused to be surfaced automatically when a determination is made by the wellness insight service that a user's anxiety level was above a threshold value during a work event. In other examples, wellness insights may be caused to be surfaced at set times and/or dates (e.g., daily, weekly, monthly, etc.). In still other examples, wellness insights may be caused to be surfaced when a selection is made to review available wellness insights. Wellness insights may be surfaced in various constructs (e.g., a productivity application, via email, in a shell construct, in pop-up windows, etc.).

The systems, methods, and devices described herein provide technical advantages for generating and surfacing wellness recommendations. Processing costs (i.e., CPU cycles) are reduced, and user efficiency is improved upon, via the mechanisms described herein at least in that users do not have to manually enter their time spent performing tasks in productivity applications to track their time spent composing and reading emails. Additionally, rather than requiring that a user manually open and review a calendar application to determine whether there is sufficient time to decompress (e.g., take a break, go for a walk), the mechanisms described herein intelligently identify when a user may be in a high anxiety state, analyze calendar application signals, and cause relevant wellness recommendations to be surfaced. Further, rather than requiring that a user individually review data from multiple applications and devices to determine an emotional state during a work event, the mechanisms described herein may automatically identify relevant data associated with work events, and analyze that data to intelligently generate anxiety scores that can be utilized to surface wellness recommendations.

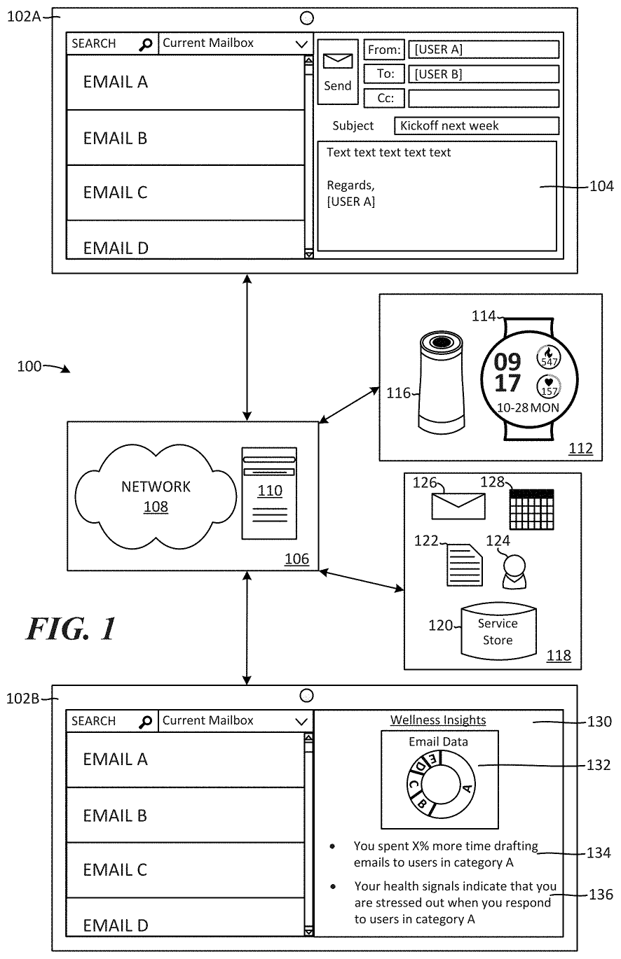

FIG. 1 is a schematic diagram illustrating an example distributed computing environment 100 for providing wellness insights in relation to a user's email. Environment 100 includes computing device 102A and computing device 102B, which may be the same or different computing devices associated with a same user account. Environment 100 also includes network and processing sub-environment 106, internet of things (IoT) sub-environment 112, and storage sub-environment 118. Any and all of the computing devices described herein may communicate with one another via a network, such as network 108.

A user account associated with user A is currently logged into computing device 102A and computing device 102B. That user account may additionally or alternatively be logged into the email application that is currently displayed on both of those devices. The email application may be executed locally by computing device 102A/102B, remotely (i.e., in the cloud), and/or partially locally and partially remotely. The user account may be associated with an email service in the cloud. In additional examples, the user account may be associated with a wellness insight service in the cloud. The email service and/or the wellness insight service may be executed by one or more server computing devices, such as server computing device 110 in network and processing sub-environment 106.

IoT sub-environment 112 includes two exemplary IoT devices that may be associated with the wellness insight service. Specifically, IoT sub-environment 112 includes smartwatch 114 and digital assistant device 116. Other wearable devices and/or IoT devices may be included in IoT sub-environment and/or associated with the wellness insight service. Such additional devices may include: fitness tracker devices, smart phone devices, tablet devices, camera devices, and/or videogame console devices, for example. User A may have authorized those IoT devices to send and/or receive data to and/or from the wellness insight service. For example, user A may utilize a settings menu associated with the IoT devices and/or the wellness insight service and specify which devices can send and/or receive data to and/or from the wellness insight service. In additional examples, the user may specify the type of data that may be sent or received by the IoT devices to and/or from the wellness insight service. In some examples, one or more of the IoT devices may communicate directly with the wellness insight service via network 108. In other examples, one or more of the IoT devices may connect directly to computing device 104 (e.g., via Bluetooth, via Wi-Fi) and communicate indirectly with the wellness insight service via computing device 104.

Storage sub-environment 118 includes service store 120, which may include stored data associated with user A's account (e.g., associated with user A's use of the email application and/or service, associated with user A's use of the wellness insight service, associated with one or more additional applications and/or services accessed from computing device 102A, associated user A's use of smartwatch 114, associated with user A's use of digital assistant device 116). In the illustrated example of storage sub-environment 118, service store 120 includes email application data 126 and/or email application metadata associated with user A; calendar application data 128 and/or calendar application metadata associated with user A; and user settings data 124, which may include user privacy settings for user A. Service store 120 may additionally or alternatively include historical biometric and/or locational data associated with smartwatch 114, and/or historical audio, video and/or digital assistant interaction data associated with digital assistant 116. In additional examples, storage sub-environment 118 may include contact information for contacts of user A. In examples, the contact information may include electronic alias information (e.g., email addresses), phone contact information, and/or location contact information. In additional examples, the contact information may include user/contact type information for user A's contacts. For example, if user A's account is associated with an enterprise, user A's contacts may be classified by hierarchical position type in the enterprise (e.g., job type hierarchy information, location type hierarchy information, proximity to user A hierarchy [based on collaboration amount, based on geographic location, based on contact overlap], etc.).

Computing device 102A displays a current mailbox of the email application in the left portion of the user interface, and email message 104, which is being composed. Email message 104 has an electronic alias for [USER A] in the “From” field, an electronic alias for [USER B] in the “To” field, and the subject “Kickoff next week” in the “Subject” field. Email message 104 further includes text that has been added by user A to the body of the email. In this example, user A may have sent email message 104 to user B. In other examples, the email may still be in the draft state (i.e., email message 104 has not yet been sent to user B).

Computing device 102A and/or the email application associated with computing device 102A may send email data associated with email message 104 to the wellness insight service in network and processing sub-environment 106. Additionally, one or more devices in IoT sub-environment 112 may send data that was obtained contemporaneously (or nearly contemporaneously) with user A's drafting of email message 104. Examples of information that may be sent by computing device 102A and/or the email application (or related applications) include: active duration of time spent on email authoring; active duration of time spent on email viewing/reading; number of times an email application/client has been manually refreshed; number of scrolls/highlights made to received emails; number of emails composed and/or sent; number of emails opened and/or read; recipient list for email; number of re-writes or corrections made during authoring of email; complexity of email (e.g., length, content type, etc.); volume of email sent on a day and/or during a temporal window; number of calendar meetings on a day and/or during a temporal window; audio data (recorded by a microphone); image data (captured by a camera); and/or rigor of key press on keyboard data (captured via haptic feedback sensors). Examples of information that may be sent by smartwatch 114 (or other IoT devices) include: heartrate values; blood pressure values; geo-location data; audio data; and image data.

In this example, the wellness insight service may receive information about email message 104. The wellness insight service may receive timestamp information that can be utilized to determine a duration of time that user A was actively drafting email message 104. The wellness insight service may receive a timestamp when email message 104 was first opened for composing and a timestamp when email message 104 was sent to user B (or manually saved as a draft). The email application may also receive timestamps corresponding to events and/or edits that occurred that were related to email message 104 (e.g., new character typed, new recipient added to “To” field). Thus, if there is a duration of time over a threshold value that passes without an event and/or edit occurring, the wellness insight service may not count that time as “active” drafting time. Therefore, the wellness insight service may be able to identify times where a user has moved on to a different task temporarily while leaving an email message open on her computing device.

The wellness insight service may receive the electronic alias information for the intended recipient of email message 104 (i.e., [USER B]). The wellness insight service may also receive geo-location data for computing device 102A and/or device identification information (e.g., device number, IP address) that may be utilized to identify a device type where the message was drafted (e.g., personal device, work device). In additional examples, the wellness insight service may receive heartrate and/or blood pressure information for user A and identify heartrate values and/or blood pressure values for times corresponding to the active authoring of email 104.

The wellness insight service may have made a determination based on the electronic alias of the recipient of email message 104 that user B is in a hierarchical category relative to user A. For example, the wellness insight service, utilizing hierarchical contact information in service store 120, may have determined that user B falls within hierarchical category A (e.g., a manager, a specific enterprise division, etc.) relative to user A. The wellness insight service may have also accessed hierarchical email and biometric information related to user A from service store 120. The additional hierarchical email information may relate to a duration of active time that user A spends drafting emails to other hierarchical category types of contacts. The additional biometric information may relate to a baseline blood pressure and/or heartrate for user A based on previously recorded blood pressure and/or heartrate data from one or more IoT devices (e.g., smartwatch 114).

Based on the information from email message 104 and the information contained in service store 120, the wellness insight service may cause one or more wellness insights to be surfaced on computing device 102B. The wellness insights may be surfaced automatically, at periodic intervals, via an email alert service, and/or or upon receiving a command to surface one or more insights. In this example, wellness insights 130 are caused to be surfaced on the right portion of computing device 102B. Wellness insights 130 include first wellness insight 132, second wellness insight 134, and third wellness insight 136.

Wellness insight 132 is a pie-chart type insight that illustrates the amount of time, by percentage, that user A spends drafting emails to users in various hierarchical categories (categories A through E). This is further illustrated by wellness insight 134, which is a text insight that states: “You spent x% more time drafting emails to users in category A”. Insight 136, which may be based on one or both of blood pressure and/or heartrate values, and historical data for those values for user A, states: “Your health signals indicate that you are stressed out when you respond to users in category A”. The stress state may be indicated by user A's heightened blood pressure and/or heartrate values during the drafting of emails to users in hierarchical category A relative to the user's heightened blood pressure and/or heartrate value during the drafting of emails to users in hierarchical categories B-D, for example.

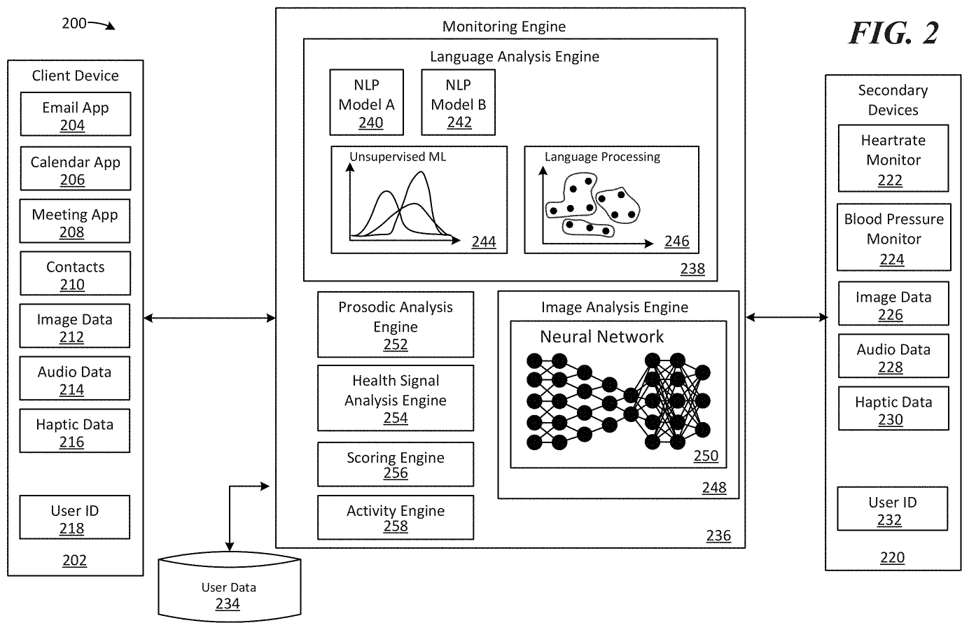

FIG. 2 illustrates a distributed computing environment 200 for generating and surfacing wellness insights in relation to user tasks. Computing environment 200 includes client device sub-environment 202, monitoring engine sub-environment 236, and secondary device sub-environment 220. Client device 202 is illustrative of a computing device that a user accesses an email application and/or service with, and on which one or more wellness insights may be surfaced on. Client device 202 may be a personal computer, a laptop, a tablet, and/or a smart phone, for example.

Client device 202 includes email application 204, calendar application 206, meeting application 208, contacts application 210, image data 212, audio data 214, haptic data 216, and user ID 218. The applications illustrated in client device 202 may be executed entirely by client device 202, entirely by one or more devices in the cloud, or partially by client device 202 and partially by one or more devices in the cloud Image data 212 represents data that may be generated via a camera that may be built into client device 202 and/or that may be connected to client device 202. For example, image data 212 may comprise images taken of a user via video or still camera. Audio data 214 represents data that may be generated via a microphone that may be built into client device 202 and/or that may be connected to client device 202. For example, audio data 214 may comprise audio recorded of a user and/or the user's surrounding environment. Haptic data 216 may comprise haptic feedback that may be generated via one or more haptic sensors that may be built into client device 202 and/or that may be connected to client device 202. For example, haptic data 216 may comprise pressure sensor data from a keyboard, mouse and/or touch screen. User ID 218 in client device 202 may comprise a user account that is utilized to sign into client device 202 and/or that is utilized to sign into and validate a user with the wellness insight service and/or one or more remote storage services.

Secondary devices 220 may comprise computing devices including one or more of: smartwatches, fitness trackers, smart phones, videogame consoles and related devices (e.g., cameras, controllers, etc.), tablets, laptops, etc. Secondary devices 220 includes heartrate monitor 222, blood pressure monitor 224, image data 226, audio data 228, and user ID 218. Heartrate monitor 222 may be an individual heart rate monitor device and/or a heartrate monitor built into a secondary device such as a smartwatch or fitness tracker. Heartrate monitor 222 may obtain heartrate values for a user, save those heartrate values, and/or transfer those values to one or more secondary storage locations. Blood pressure monitor 224 may be an individual blood pressure monitor device and/or a blood pressure monitor built into a secondary device such as a smartwatch or fitness tracker. Blood pressure monitor 224 may obtain blood pressure values for a user, save those blood pressure values, and/or transfer those values to one or more secondary storage locations.

Image data 226 represents data that may be generated via a camera that may be built into secondary devices 220 and/or that may be connected to secondary devices 220. For example, image data 226 may comprise images taken of a user via a video or still camera. Audio data 228 represents data that may be generated via a microphone that may be built into secondary devices 220 and/or that may be connected to secondary devices 220. For example, audio data 228 may comprise audio recorded of a user and/or the user's surrounding environment. Haptic data 230 may comprise haptic feedback that may be generated via one or more haptic sensors that may be built into secondary devices 220 and/or that may be connected to secondary devices 220. For example, haptic data 220 may comprise pressure sensor data from a keyboard, mouse and/or touch screen. User ID 232 in secondary devices 220 may comprise a user account that is utilized to sign into secondary devices 220 and/or that is utilized to sign into and validate a user with the wellness insight service and/or one or more remote storage services. User ID 232 may be the same as or different from user ID 218.

One or more components of monitoring engine 236 may be included in the wellness insight service. One or more components of monitoring engine 236 may additionally or alternatively be included in client device 202 and/or secondary devices 220.

Activity engine 258 receives activity signals associated with an email application, computing device executing an email application, and/or one or more secondary devices. In some examples, activity engine 258 may determine whether a user is actively engaged with the composing and/or reading of an email. For example, activity engine 258 may receive data indicating that a user is typing in an email, adding contacts to an email, and/or scrolling in an email, and therefore determine that the user is actively engaged in composing the email. In other examples, activity engine 258 may receive data indicating that, although an email message is open, the user is actively engaged with a different application. As such, activity engine 258 may make a determination that the user is not actively engaged with the email. In additional examples, activity engine 258 may determine that a user is actively engaged in reading an email because the email is being scrolled through and/or highlights are being made to the email In additional examples, activity engine 258 may utilize a camera associated with client device 202 and/or secondary device 220 and utilize a gaze detection engine to determine whether a user is actively engaged with the composing and/or reading of an email.

Language analysis engine 238 includes NLP (natural language processing) model A 240, NLP model B 242, unsupervised machine learning model 244 and language processing element 246. Unsupervised machine learning model 244 and language processing element 246 illustrate that various language modeling types and/or machine learning models may be utilized to process language input into monitoring engine 236. NLP model A 240 may analyze language from emails sent or received by a user account associated with client device 202 (e.g., associated with user ID 218). NLP model A 240 may be trained to identify a level of complexity associated with natural language and/or to classify language into tone categories (e.g., angry, happy, indifferent, etc.). NLP model B 242 may also analyze language from emails sent or received by a user account associated with client device 202 (e.g., associated with user ID 218). NLP model B 242 may be trained to identify one or more task intents and/or task relationships (e.g., perform X action, assign task A to user B, etc.) associated with natural language, identify one or more contacts mentioned in natural language, and/or identify one or more objects (e.g., documents, files, etc.) mentioned in natural language.

Prosodic analysis engine 252 may analyze audio data received from client device 202 and/or secondary devices 220. Prosodic analysis engine 252 may include one or more audio processing models that have been trained to identify an anxiety or stress level of a user. In some examples, in identifying an anxiety or stress level, one or more audio processing models may compare a specific user's voice data against a baseline that has been generated for that specific user utilizing historic audio voice data for that user. In other examples, in identifying an anxiety or stress level, one or more audio processing models may compare a specific user's voice data against a baseline that has been generated from other users' voice data.

Health signal analysis engine 254 may analyze one or more biometric signals (e.g., heartrate values, blood pressure values) from secondary devices 220. Health signal analysis engine 254 may determine whether a user's heartrate values and/or blood pressure values are above a baseline value (e.g., a specific user may have a resting heartrate and/or blood pressure that is higher or lower than an ideal and/or average for other users). A baseline value may be specific to the user or a baseline value may be based on a baseline for other users.

Image analysis engine 248 may analyze one or more images of a user and identify one or more emotional state types associated with the user. The images may be received from client device 202 and/or secondary devices 220. The images may comprise facial images, torso images, and/or facial and torso images. Image analysis engine includes neural network 250, which is illustrative of an exemplary model that may be included in image analysis engine 248 for classifying user images into emotional state types (e.g., happy, anxious, stressed, sad, etc.).

User data 234 may comprise account data associated with user ID 218 and/or user ID 232. User data 234 may include email data (sent and received emails and related metadata), contacts information, hierarchical enterprise information, historical biometric data (e.g., historical heartrate and/or blood pressure data), and/or historical image, audio and/or haptic data for a user associated with one or both of ID 218 and/or user ID 232. User data 234 may be utilized by any of the models described herein for training and or baseline comparison purposes.

Scoring engine 256 may receive data from one or more of: language analysis engine 238, prosodic analysis engine 252, health signal analysis engine 254, and/or image analysis engine 248, and generate an emotional score for a user and a corresponding task event based on that data. A task event may comprise an email composing event, an email reviewing event, and/or a meeting event. In some examples, each of language analysis engine 238, prosodic analysis engine 252, health signal analysis engine 254, and/or image analysis engine 248 may have their own scoring engines, and scoring engine 256 may generate a final emotional score for a task event based on the combination of those scores. For example, a complexity score and/or one or more task intent scores may be generated by language analysis engine 238 for an email, one or more scores corresponding emotional states may be generated by prosodic analysis engine for a meeting event, one or more scores for heartrate and/or blood pressure for a user during an email event may be generated by health signal analysis engine 254, and one or more scores may be generated by image analysis engine 248 for one or more images based on their relationship/classification to an emotional state type for a user during an email and/or meeting event. Various scoring techniques may be utilized by scoring engine 256 in generating a final emotional score for a task event. In some examples, scoring engine 256 may apply weights to one or more scores generated by the other engines (e.g., a score for one engine may have a higher weight assigned to it than a score for a different engine).

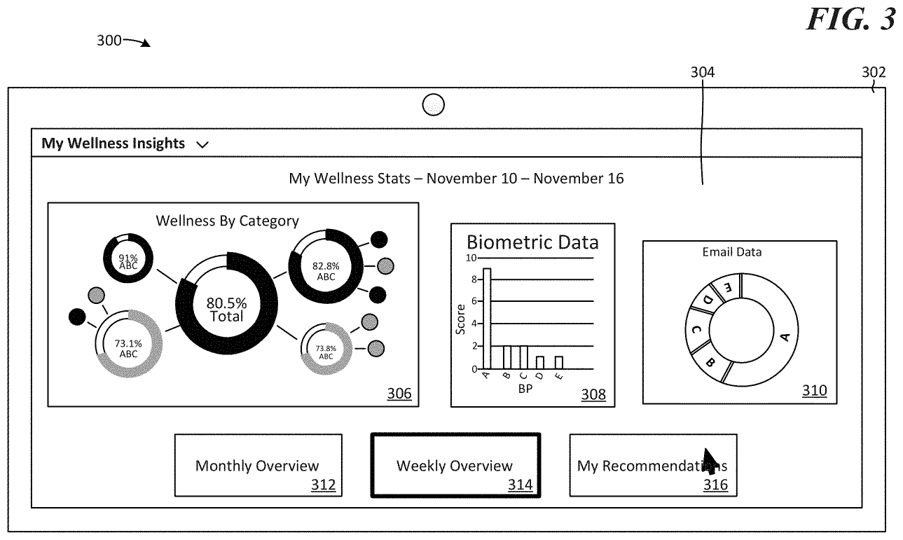

FIG. 3 illustrates a computing environment, including a dashboard user interface 304, for surfacing wellness insights. Dashboard user interface 304 is displayed on computing device 302. A user may have signed into computing device 302 and/or one or more applications executed by computing device 302 (or web applications executed via computing device 302) utilizing a user ID and password. Once authenticated, the user ID may provide access to information associated with the wellness insight service.

The information displayed on user interface 304 may be associated with a single user account and corresponding user ID. That information may be caused to be surfaced based on receiving an indication to present wellness insights. In other examples, that information may be caused to be surfaced automatically. For example, the displayed wellness insight information may be surfaced automatically based on a triggering event occurring. The triggering event may comprise the sending of an email, the receiving of an email, a designated time and/or day occurring (e.g., an end of day report, an end of week report), etc.

User interface 304 displays exemplary wellness insights for a user based on data received between November 10 and November 16. The wellness insights include wellness by category graph 306, biometric data table 308, and email data pie chart 310.

Wellness by category graph 306 illustrates a percentage of a user's wellness that each of a plurality of work tasks contribute to (illustrated by circular graph object size), and the total realized wellness out of one-hundred percent that a user experienced for each task type (illustrated by a fill bar on the outside of a circular graph object). For example, the upper left circular graph object may represent the composing of emails to close colleagues, and a user may realize a healthy 91% wellness score for that category. Alternatively, the lower left circular graph object may represent the reviewing of manager emails, and a user may realize a 73% wellness score for that category, illustrating that it could use some work both because it contributes more to the user's overall wellness than the composing of emails to close colleagues, and because the wellness score is lower for that category.

Biometric data table 308 may illustrate a user's blood pressure values while performing various task types (task type A, task type B, task type C, task type D, task type E), or the user's blood pressure values while drafting or reviewing emails to different categories of users (e.g., category A, category B, category C, category D, category E). In this example, the user's blood pressure is illustrated as being much higher in relation to task type A or category A.

Email data pie chart 310 may illustrate a generated anxiety score for a user during the drafting and/or reviewing of emails to different categories of users (category A, category B, category C, category D, category E). Similar to biometric data table 308, email data pie chart 310 indicates that the user's anxiety score is much higher for category A than the other categories.

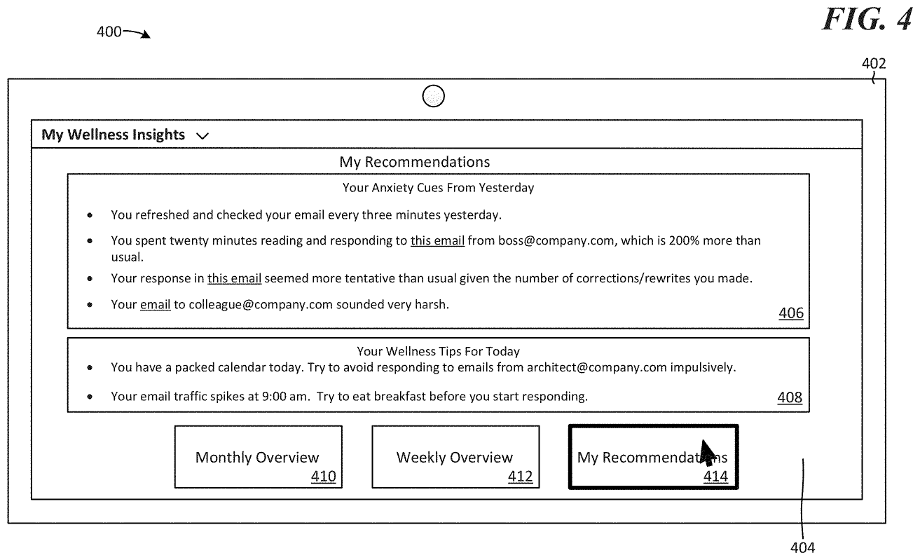

User interface 304 also includes monthly overview element 312, weekly overview element 314, and my recommendations element 316. In this example, weekly overview 314 is currently selected, which is why the wellness stats for November 10-November 16 are currently displayed. Monthly stats may be generated and caused to be surfaced upon receiving a selection of monthly overview element 312. In this example, a mouse cursor is hovered over my recommendations element 316. The selection of my recommendations element 316 may result in the surfacing of elements discussed below in relation to FIG. 4.

FIG. 4 illustrates a computing environment 400 for surfacing wellness recommendations related to users' task and health signals. My recommendations dashboard user interface 404 is displayed on computing device 402 in response to selection of my recommendations element 414. The recommendations dashboard includes an explanation of a plurality of events that the wellness insight service identified as causing a heightened degree of anxiety for the user. The recommendations dashboard also includes a plurality of tips for reducing a user's anxiety and increasing wellness.

The explanation of the plurality of events that the wellness insight service identified as causing a heightened degree of anxiety for the user are included in anxiety cues window 406. Anxiety cues window 406 includes the heading “Your Anxiety Cues From Yesterday”. Anxiety cues window 406 includes a first cue, which states: “You refreshed and checked your email every three minutes yesterday.” Anxiety cues window 406 includes a second cue, which states: “You spent twenty minutes reading and responding to this email from boss@company.com , which is 200% more than usual.” The phrase “this email” in the second cue may be selectable for surfacing the corresponding email on computing device 402. Anxiety cues window 406 includes a third cue, which states: “Your response in this email seemed more tentative than usual given the number of corrections/rewrites you made.” The phrase “this email” in the third cue may be selectable for surfacing the corresponding email on computing device 402. Anxiety cues window 406 includes a fourth cue, which states: “Your email to colleague@company.com sounded very harsh.” The “harshness” of the email may have been identified by the wellness insight service via one or more natural language processing models. The word “email” in the fourth cue may be selectable for surfacing the corresponding email on computing device 402.

The plurality of tips for reducing a user's anxiety and increasing wellness are included in tips window 408. Tips window 408 includes a first tip, which states: “You have a packed calendar today. Try to avoid responding to emails from architect@company.com impulsively.” The wellness insight service may have reviewed calendar signals from the user's electronic calendar in generating this tip. Additionally, the wellness insight service may have identified that the user has a higher level of anxiety in relation to composing and/or reviewing emails to/from architect@company.com. Tips window 408 also includes a second tip, which states: “Your email traffic spikes at 9:00 am. Try to eat breakfast before you start responding.”

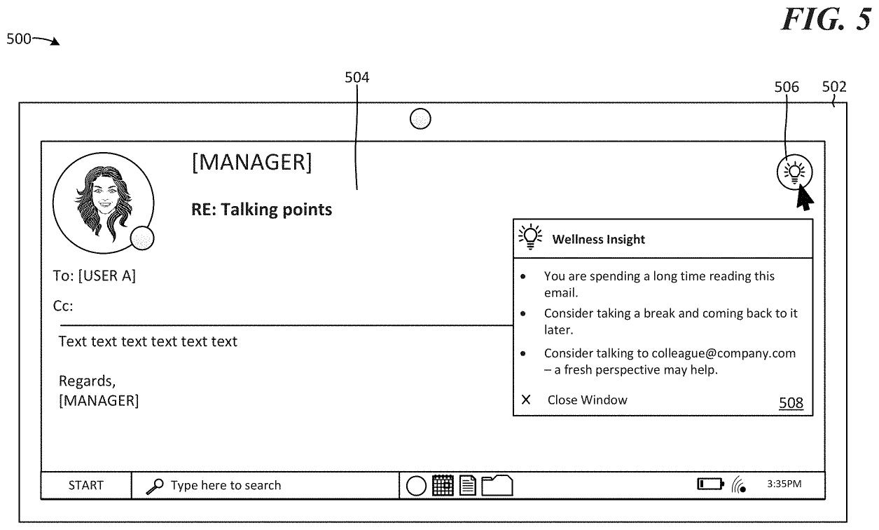

FIG. 5 illustrates a computing environment 500 for surfacing wellness insights in relation to an email 504 that has been received. Computing environment 500 includes computing device 502. Computing device 502 displays email 504, which was sent by [MANAGER] to [USER A]. In this example, the insight wellness service has caused insight element 506 to be displayed in association with email 504. Insight element 506 indicates that the wellness insight service has identified one or more wellness insights for a user. In this example, the wellness insight service may have analyzed an active amount of time that user A is spending, or spent, reading email 504, a relationship of user A to the sender, and/or one or more contacts of user A that may be helpful in completing a task associated with email 504.

In the current example, a selection of insight element 506 has been made. Wellness insight window 508 is then caused to be surfaced in association with email 504. Wellness insight window 508 includes a first insight, which states: “You are spending a long time reading this email.” Wellness insight window 508 includes a second insight, which states: “Consider taking a break and coming back to it later.” Wellness insight window 508 includes a third insight, which states: “Consider talking to colleague@company.com—a fresh perspective may help.” Other suggestions/insights may be surfaced in relation to determining that a user is spending above a threshold amount of time reviewing an email.

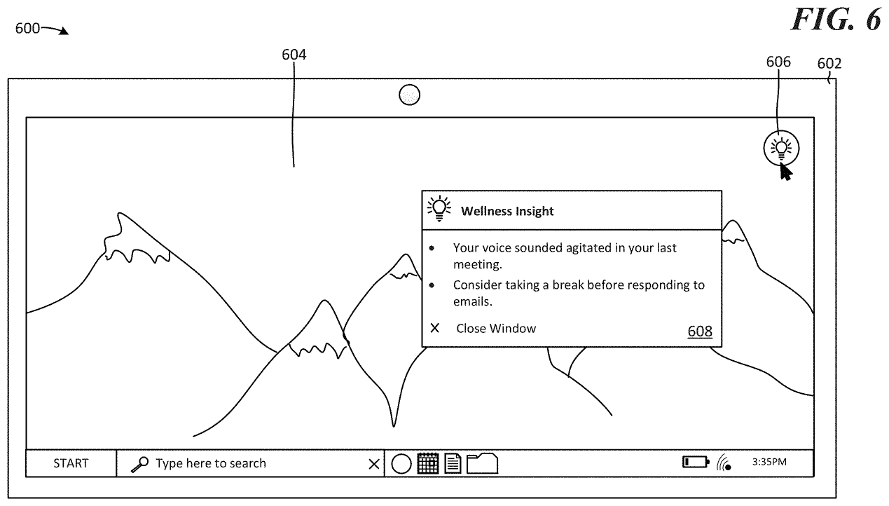

FIG. 6 illustrates a computing environment 600 for surfacing wellness insights in relation to a meeting. Computing environment 600 includes computing device 602. Computing device 602 displays desktop 604. In this example, the insight wellness service has caused insight element 606 to be displayed in association with desktop 604. Insight element 606 indicates that the wellness insight service has identified one or more wellness insights for a user. In this example, the wellness insight service may have analyzed lexical and/or prosodic features of a user's voice (e.g., via audio data from a microphone integrated in computing device 602 or a secondary device). The wellness insight service may have identified that the user was in a meeting during part of an audio recording by analyzing the user's electronic calendar, one or more emails, and/or by analyzing natural language inputs via audio recording, for example.

In the current example, a selection of insight element 606 has been made. Wellness insight window 608 is then caused to be surfaced in association with desktop 604. Wellness insight window 608 includes a first insight, which states: “Your voice sounded agitated in your last meeting.” Wellness insight window 508 includes a second insight, which states: “Consider taking a break before responding to emails.” Other suggestions/insights may be surfaced in relation to determining that a user's voice sounded agitated and/or anxious to above a threshold value.

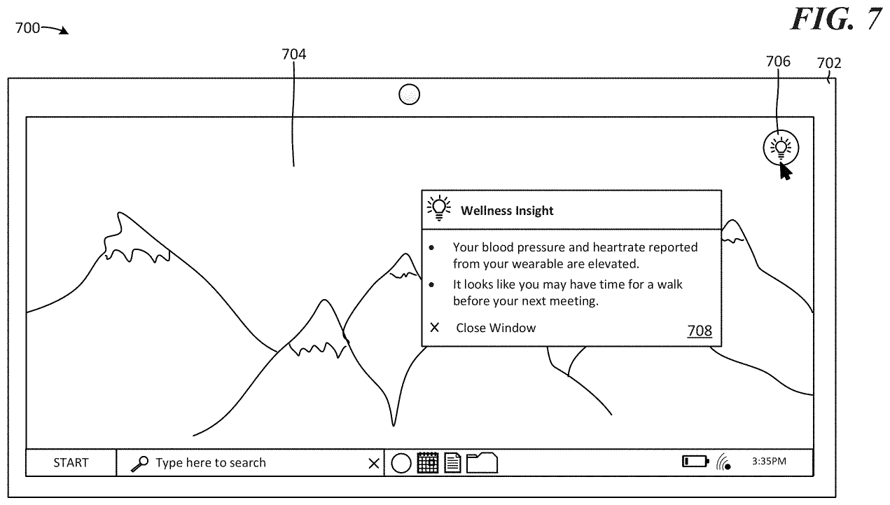

FIG. 7 illustrates a computing environment 700 for surfacing wellness insights in relation to a calendar application and health signals from a wearable device. Computing environment 700 includes computing device 702. Computing device 702 displays desktop 704. In this example, the insight wellness service has caused insight element 706 to be displayed in association with desktop 704. Insight element 706 indicates that the wellness insight service has identified one or more wellness insights for a user. In this example, the wellness insight service may have analyzed blood pressure values and heart rate values for a user based on data received from a wearable device. The wellness insight service may have also analyzed a user's electronic calendar to determine whether the user has sufficient time to take a break and go for a walk. In other examples, the wellness insight service may analyze the user's electronic calendar to determine whether the user has sufficient time to perform other wellness activities (e.g., workout, do yoga, do breathing exercises, etc.). In still additional examples, the wellness insight service may receive and/or obtain information from third-party services to determine times that wellness activities may be accessed (e.g., analyze a gym class schedule from a user's gym, analyze a cafeteria's schedule do determine if it is open, etc.).

In the current example, a selection of insight element 706 has been made. Wellness insight window 708 is then caused to be surfaced in association with desktop 704. Wellness insight window 708 includes a first insight, which states: “Your blood pressure and heartrate reported from your wearable are elevated.” Wellness insight window 708 includes a second insight, which states: “It looks like you may have time for a walk before your next meeting.” Other suggestions/insights may be surfaced in relation to determining that a user's blood pressure and/or heartrate is higher than a baseline (or above a threshold value of a baseline).

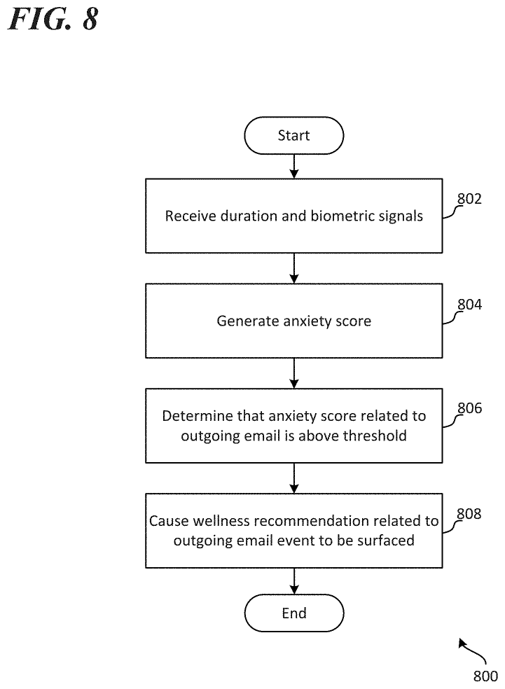

FIG. 8 is an exemplary method 800 for surfacing wellness insights related to an outgoing email. The method 800 begins at a start operation and flow moves to operation 802.

At operation 802 a plurality of signals related to a user are received. The plurality of signals may be received from one or more applications associated with a user account. The applications may be installed locally on a user's client computing device or they may be web applications. The plurality of signals may additionally or alternatively be received from one or more components of a user's client computing device (e.g., a camera, a speaker, etc.). In additional examples, the plurality of signals may be received from one or more secondary devices (e.g., heartrate monitor, blood pressure monitor, smartwatch, digital assistant device, etc.).

The plurality of signals may comprise an active duration of time spent composing an outgoing email sent from a user account associated with the user. The active duration may be identified based on the email being interacted with in the email application (e.g., words typed in email, mouse moved in email, recipients added to email) and/or one or more user cues (e.g., based on user gaze detection, based on a user authoring email via dictation). The plurality of signals may further comprise a biometric signal associated with the user comprising at least one of: a blood pressure value for the user during a time that the outgoing email was being composed, and a heartrate value during a time that the outgoing email was being composed. In additional examples, the plurality of signals may comprise a natural language input included in the outgoing email. In some examples, a natural language processing model may be applied to the natural language input. The natural language processing model may have been trained to classify natural language inputs into tone categories (e.g., angry tone, happy tone, indifferent tone, etc.) and/or complexity categories.

From operation 802 flow continues to operation 804 where an anxiety score associated with the outgoing email is generated for the user. In some examples, the anxiety score may be generated based on an algorithm. In additional examples, the anxiety score may be generated based on a weighted system (e.g., one signal may have more weight assigned to it than another signal). In still additional examples, the anxiety score may be generated via application of one or more machine learning models to the signals.

From operation 804 flow continues to operation 806 where a determination is made that the anxiety score is above a threshold baseline value for the user. In some examples, the baseline may be based on historical signal data for the user. In other examples, the baseline may be based on historical data for a plurality of users (e.g., data from randomly identified users, data from users that have overlapping demographics, etc.). According to additional examples, the threshold baseline value may be a baseline for emails the user sends to a recipient account that the outgoing email is addressed to. For example, the user may have a baseline anxiety level associated with sending emails to managers that is less than a baseline anxiety level associated with sending emails to her family.

From operation 806 flow continues to operation 808 where a wellness recommendation related to the outgoing email event is caused to be surfaced. The wellness recommendation may include an explanation of one or more signals that were identified as potentially causing anxiety. The wellness recommendation may additionally or alternatively include one or more suggestions for reducing anxiety.

From operation 808 flow moves to an end operation and the method 800 ends.

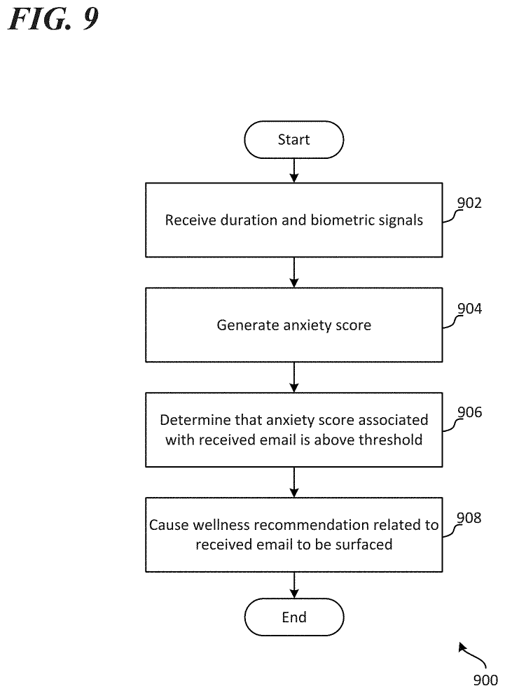

FIG. 9 is an exemplary method 900 for surfacing wellness insights related to a received email. The method 900 begins at a start operation and flow moves to operation 902.

At operation 902 a plurality of signals related to a user are received. The plurality of signals may be received from one or more applications associated with a user account. The applications may be installed locally on a user's client device computing device or they may be web applications. The plurality of signals may additionally or alternatively be received from one or more components of a user's client computing device (e.g., a camera, a speaker, etc.). In additional examples, the plurality of signals may be received from one or more secondary devices (e.g., heartrate monitor, blood pressure monitor, smartwatch, digital assistant device, etc.).

The plurality of signals may comprise an active duration of time spend reviewing a received email. The active duration may be identified based on the email being interacted with in the email application (e.g., receiving scroll commands associated with the email, receiving highlight commands of text in the email, based on receiving commands in other applications, etc.) and/or one or more user cues (e.g., based on user gaze detection). The plurality of signals may additionally comprise a biometric signal associated with the user comprising at least one of: a blood pressure value for the user during a time that the received email was open in an email application associated with the user, and a heartrate value for the user during a time that the received email was open in an email application associated with the user. The plurality of signals may further comprise a natural language input included in the email. In some examples, a natural language processing model may be applied to the natural language input. The natural language processing model may have been trained to classify natural language inputs into tone categories (e.g., angry tone, happy tone, indifferent tone, etc.) and/or complexity categories.

From operation 902 flow continues to operation 904 where an anxiety score associated with the received email is generated. In some examples, the anxiety score may be generated based on an algorithm. In additional examples, the anxiety score may be generated based on a weighted system (e.g., one signal may have more weight assigned to it than another signal). In still additional examples, the anxiety score may be generated via application of one or more machine learning models to the signals.

From operation 904 flow continues to operation 906 where a determination is made as to whether the anxiety score is above a threshold baseline value for the user. In some examples, the baseline may be based on historical signal data for the user. In other examples, the baseline may be based on historical data for a plurality of users (e.g., data from randomly identified users, data from users that have overlapping demographics, etc.). According to additional examples, the threshold baseline value may be a baseline for emails the user receives from a specific sender. For example, the user may have a baseline anxiety level associated with receiving/reading emails from a manager that is higher than for emails received from family members or other coworkers.

From operation 906 flow continues to operation 908 where a wellness recommendation related to the received email is caused to be surfaced. The wellness recommendation may include an explanation of one or more signals that were identified as potentially causing anxiety. The wellness recommendation may additionally or alternatively include one or more suggestions for reducing anxiety.

From operation 908 flow moves to an end operation and the method 900 ends.

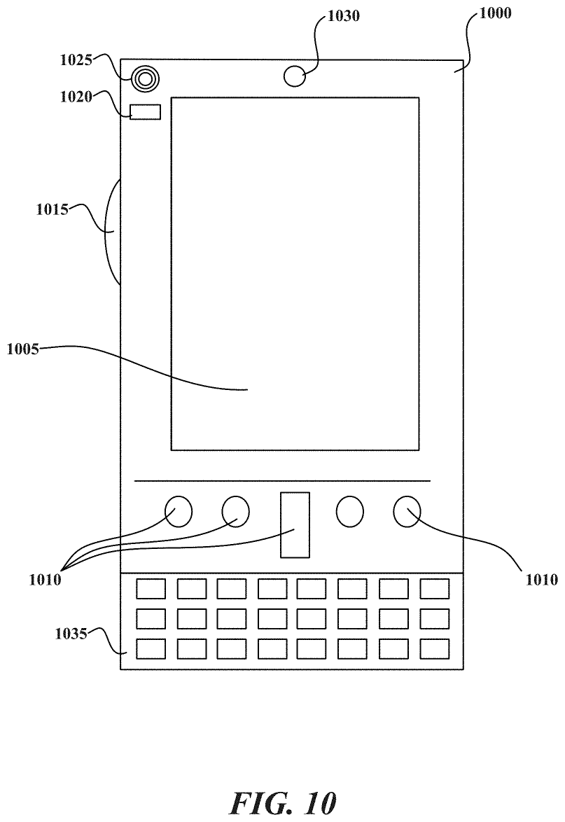

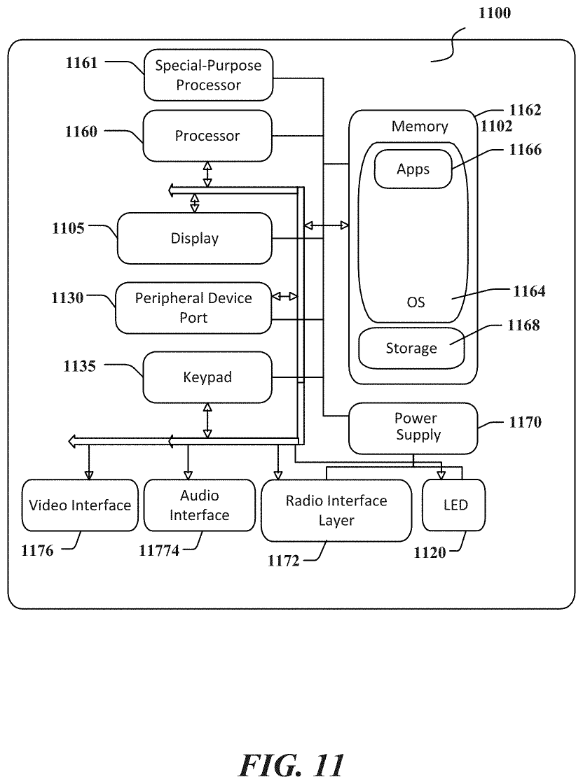

FIGS. 10 and 11 illustrate a mobile computing device 1000, for example, a mobile telephone, a smart phone, wearable computer (such as smart eyeglasses, a smartwatch, a fitness tracker), a tablet computer, an e-reader, a laptop computer, or other AR compatible computing device, with which embodiments of the disclosure may be practiced. With reference to FIG. 10, one aspect of a mobile computing device 1000 for implementing the aspects is illustrated. In a basic configuration, the mobile computing device 1000 is a handheld computer having both input elements and output elements. The mobile computing device 1000 typically includes a display 1005 and one or more input buttons 1010 that allow the user to enter information into the mobile computing device 1000. The display 1005 of the mobile computing device 1000 may also function as an input device (e.g., a touch screen display). If included, an optional side input element 1015 allows further user input. The side input element 1015 may be a rotary switch, a button, or any other type of manual input element. In alternative aspects, mobile computing device 1000 may incorporate more or fewer input elements. For example, the display 1005 may not be a touch screen in some embodiments. In yet another alternative embodiment, the mobile computing device 1000 is a portable phone system, such as a cellular phone. The mobile computing device 1000 may also include an optional keypad 1035. Optional keypad 1035 may be a physical keypad or a “soft” keypad generated on the touch screen display. In various embodiments, the output elements include the display 1005 for showing a graphical user interface (GUI), a visual indicator 1020 (e.g., a light emitting diode), and/or an audio transducer 1025 (e.g., a speaker). In some aspects, the mobile computing device 1000 incorporates a vibration transducer for providing the user with tactile feedback. In yet another aspect, the mobile computing device 1000 incorporates input and/or output ports, such as an audio input (e.g., a microphone jack), an audio output (e.g., a headphone jack), and a video output (e.g., a HDMI port) for sending signals to or receiving signals from an external device.

FIG. 11 is a block diagram illustrating the architecture of one aspect of a mobile computing device. That is, the mobile computing device 1100 can incorporate a system (e.g., an architecture) 1102 to implement some aspects. In one embodiment, the system 1102 is implemented as a “smart phone” capable of running one or more applications (e.g., browser, e-mail, calendaring, contact managers, messaging clients, games, and media clients/players). In some aspects, the system 1102 is integrated as a computing device, such as an integrated personal digital assistant (PDA) and wireless phone.

One or more application programs 1166 may be loaded into the memory 1162 and run on or in association with the operating system 1164. Examples of the application programs include phone dialer programs, e-mail programs, personal information management (PIM) programs, word processing programs, spreadsheet programs, Internet browser programs, messaging programs, and so forth. The system 1102 also includes a non-volatile storage area 1168 within the memory 1162. The non-volatile storage area 1168 may be used to store persistent information that should not be lost if the system 1102 is powered down. The application programs 1166 may use and store information in the non-volatile storage area 1168, such as e-mail or other messages used by an e-mail application, and the like. A synchronization application (not shown) also resides on the system 1102 and is programmed to interact with a corresponding synchronization application resident on a host computer to keep the information stored in the non-volatile storage area 1168 synchronized with corresponding information stored at the host computer. As should be appreciated, other applications may be loaded into the memory 1162 and run on the mobile computing device 1100, including instructions for providing and operating a digital assistant computing platform.

The system 1102 has a power supply 1170, which may be implemented as one or more batteries. The power supply 1170 might further include an external power source, such as an AC adapter or a powered docking cradle that supplements or recharges the batteries.

The system 1102 may also include a radio interface layer 1172 that performs the function of transmitting and receiving radio frequency communications. The radio interface layer 1172 facilitates wireless connectivity between the system 702 and the “outside world,” via a communications carrier or service provider. Transmissions to and from the radio interface layer 1172 are conducted under control of the operating system 1164. In other words, communications received by the radio interface layer 1172 may be disseminated to the application programs 1166 via the operating system 1164, and vice versa.

The visual indicator 1020 may be used to provide visual notifications, and/or an audio interface 1174 may be used for producing audible notifications via the audio transducer 1025. In the illustrated embodiment, the visual indicator 1020 is a light emitting diode (LED) and the audio transducer 1025 is a speaker. These devices may be directly coupled to the power supply 1170 so that when activated, they remain on for a duration dictated by the notification mechanism even though the processor 1160 and other components might shut down for conserving battery power. The LED may be programmed to remain on indefinitely until the user takes action to indicate the powered-on status of the device. The audio interface 1174 is used to provide audible signals to and receive audible signals from the user. For example, in addition to being coupled to the audio transducer 1025, the audio interface 1174 may also be coupled to a microphone to receive audible input, such as to facilitate a telephone conversation. In accordance with embodiments of the present disclosure, the microphone may also serve as an audio sensor to facilitate control of notifications, as will be described below. The system 1102 may further include a video interface 1176 that enables an operation of an on-board camera 1030 to record still images, video stream, and the like.

A mobile computing device 1100 implementing the system 1102 may have additional features or functionality. For example, the mobile computing device 1100 may also include additional data storage devices (removable and/or non-removable) such as, magnetic disks, optical disks, or tape. Such additional storage is illustrated in FIG. 11 by the non-volatile storage area 1168.

Data/information generated or captured by the mobile computing device 1100 and stored via the system 1102 may be stored locally on the mobile computing device 1100, as described above, or the data may be stored on any number of storage media that may be accessed by the device via the radio interface layer 1172 or via a wired connection between the mobile computing device 1100 and a separate computing device associated with the mobile computing device 1100, for example, a server computer in a distributed computing network, such as the Internet. As should be appreciated such data/information may be accessed via the mobile computing device 1100 via the radio interface layer 1172 or via a distributed computing network. Similarly, such data/information may be readily transferred between computing devices for storage and use according to well-known data/information transfer and storage means, including electronic mail and collaborative data/information sharing systems.

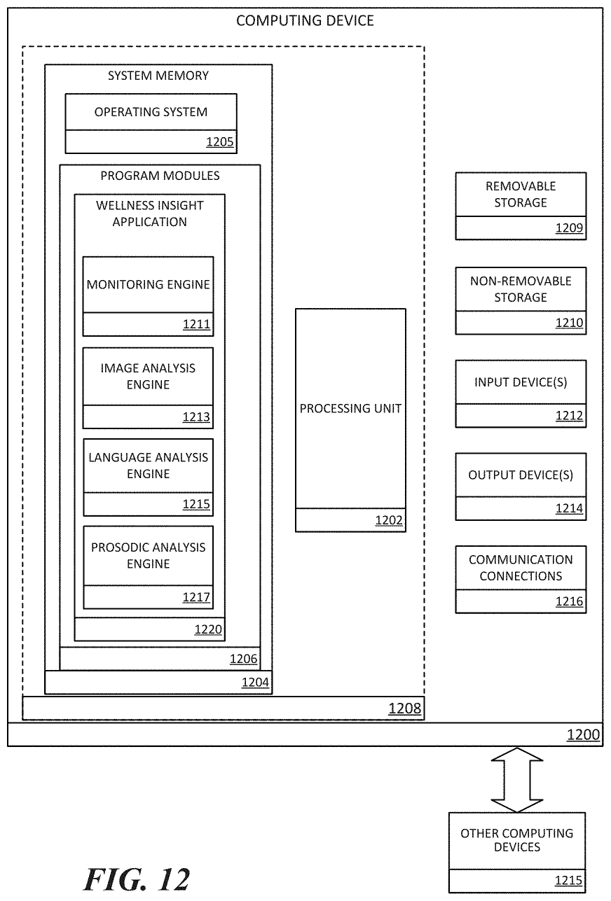

FIG. 12 is a block diagram illustrating physical components (e.g., hardware) of a computing device 1200 with which aspects of the disclosure may be practiced. The computing device components described below may have computer executable instructions for generating, surfacing and providing operations associated with wellness insights. In a basic configuration, the computing device 1200 may include at least one processing unit 1202 and a system memory 1204. Depending on the configuration and type of computing device, the system memory 1204 may comprise, but is not limited to, volatile storage (e.g., random access memory), non-volatile storage (e.g., read-only memory), flash memory, or any combination of such memories. The system memory 1204 may include an operating system 1205 suitable for running one or more wellness insight programs. The operating system 1205, for example, may be suitable for controlling the operation of the computing device 1200. Furthermore, embodiments of the disclosure may be practiced in conjunction with a graphics library, other operating systems, or any other application program and is not limited to any particular application or system. This basic configuration is illustrated in FIG. 12 by those components within a dashed line 1208. The computing device 1200 may have additional features or functionality. For example, the computing device 1200 may also include additional data storage devices (removable and/or non-removable) such as, for example, magnetic disks, optical disks, or tape. Such additional storage is illustrated in FIG. 12 by a removable storage device 1209 and a non-removable storage device 1210.

As stated above, a number of program modules and data files may be stored in the system memory 1204. While executing on the processing unit 1202, the program modules 1206 (e.g., wellness insight application 1220) may perform processes including, but not limited to, the aspects, as described herein. According to examples, monitoring engine 1211 may perform one or more operations associated with identifying cues from signals to determine an anxiety level associated with an email sender/recipient. In examples, monitoring engine 1211 may log those cues for offline reporting to the user if the user has opted for such a service. In additional examples, monitoring engine 1211 may send real-time feedback via soothing notifications or nudges to the user's client device to coach/reduce anxiety levels Image analysis engine 1213 may perform one or more operations associated with analyzing user images (e.g., facial images) and classifying those images into emotional state types. Language analysis engine 1215 may perform one or more operations associated with analyzing lexical features of written and/or spoken language from a user and classifying that language into categories. The categories may be based on complexity and/or emotional state. Prosodic analysis engine 1217 may perform one or more operations associated with analyzing prosodic features in voice data and classifying that data based on emotional state (e.g., angry, stressed, anxious, etc.).

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 12 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 1200 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general purpose computer or in any other circuits or systems.

The computing device 1200 may also have one or more input device(s) 1212 such as a keyboard, a mouse, a pen, a sound or voice input device, a touch or swipe input device, etc. The output device(s) 1214 such as a display, speakers, a printer, etc. may also be included. The aforementioned devices are examples and others may be used. The computing device 1200 may include one or more communication connections 1216 allowing communications with other computing devices 1250. Examples of suitable communication connections 1216 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 1204, the removable storage device 1209, and the non-removable storage device 1210 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 1200. Any such computer storage media may be part of the computing device 1200. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

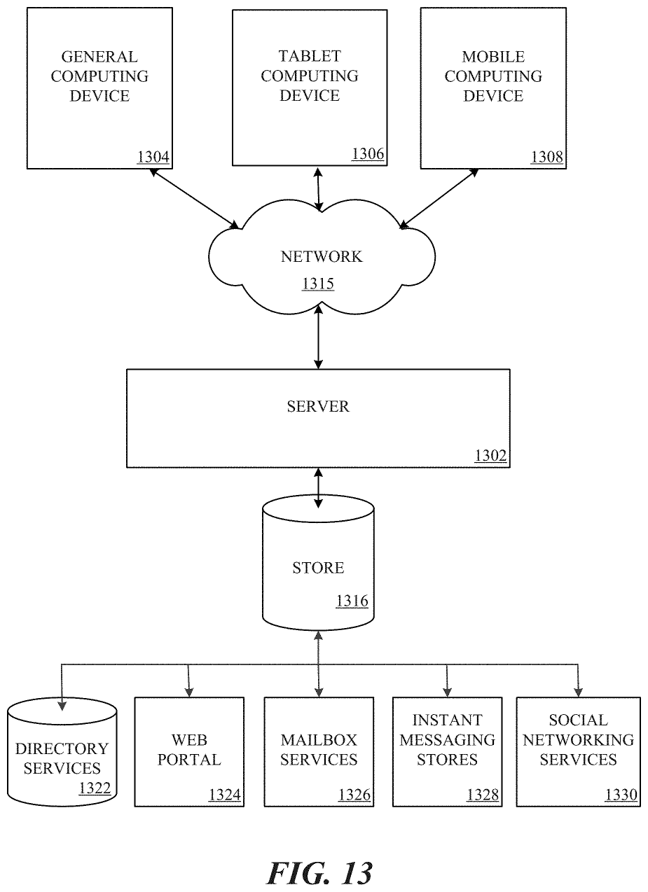

FIG. 13 illustrates one aspect of the architecture of a system for processing data received at a computing system from a remote source, such as a personal/general computer 1304, tablet computing device 1306, or mobile computing device 1308, as described above. Content displayed at server device 1302 may be stored in different communication channels or other storage types. For example, various documents may be stored using a directory service 1322, a web portal 1324, a mailbox service 1326, an instant messaging store 1328, or a social networking site 1330. The program modules 1206 may be employed by a client that communicates with server device 1302, and/or the program modules 1206 may be employed by server device 1302. The server device 1302 may provide data to and from a client computing device such as a personal/general computer 1304, a tablet computing device 1306 and/or a mobile computing device 1308 (e.g., a smart phone) through a network 1315. By way of example, the computer systems described herein may be embodied in a personal/general computer 1304, a tablet computing device 1306 and/or a mobile computing device 1308 (e.g., a smart phone). Any of these embodiments of the computing devices may obtain content from the store 1316, in addition to receiving graphical data useable to be either pre-processed at a graphic-originating system, or post-processed at a receiving computing system.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present disclosure, one skilled in the art may envision variations, modifications, and alternate aspects falling within the spirit of the broader aspects of the general inventive concept embodied in this application that do not depart from the broader scope of the claimed disclosure.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A computer-implemented method for surfacing wellness recommendations, the method comprising:
receiving a plurality of signals related to a user, the plurality of signals comprising:
an active duration of time spent composing an outgoing email sent from a user account associated with the user, and
a biometric signal associated with the user comprising at least one of:
a blood pressure value for the user during a time that the outgoing email was being composed, and
a heartrate value during a time that the outgoing email was being composed;
generating an anxiety score associated with the outgoing email for the user;
determining that the anxiety score is above a threshold baseline value for the user; and
causing a wellness recommendation related to the outgoing email to be surfaced.

2. The computer-implemented method of claim 1, wherein the plurality of signals further comprises a natural language input included in the outgoing email.

3. The computer-implemented method of claim 2, further comprising:
applying a natural language processing model to the natural language input, wherein the natural language processing model has been trained to classify natural language inputs into tone categories.

4. The computer-implemented method of claim 1, wherein the threshold baseline value is a baseline for emails the user sends to a recipient account that the outgoing email is addressed to.

5. The computer-implemented method of claim 1, wherein the plurality of signals further comprises at least one of:
a number of word changes made to the outgoing email while being composed;
a number of word deletions made to the outgoing email while being composed; and
a number of character deletions made to the outgoing email while being composed.

6. The computer-implemented method of claim 1, wherein the biometric signal associated with the user further comprises:
an image of facial features of the user taken during a time that the outgoing email was being composed.

7. The computer-implemented method of claim 6, wherein generating the anxiety score further comprises:
applying a neural network to the image, wherein the neural network has been trained to classify facial feature images into expression type categories.

8. The computer-implemented method of claim 1, wherein the plurality of signals further comprises a haptic signal from a keyboard while the outgoing email was being composed.

9. The computer-implemented method of claim 8, wherein the haptic signal is a pressure signal.

10. A system for surfacing wellness recommendations, comprising:
a memory for storing executable program code; and
one or more processors, functionally coupled to the memory, the one or more processors being responsive to computer-executable instructions contained in the program code and operative to:
receive a plurality of signals related to a user, the plurality of signals comprising:
an active duration of time spent reviewing a received email, and
a biometric signal associated with the user comprising at least one of:
a blood pressure value for the user during a time that the received email was open in an email application associated with the user, and
a heartrate value for the user during a time that the received email was open in an email application associated with the user;
generate an anxiety score associated with the received email;
determine that the anxiety score is above a threshold baseline value for the user; and
cause a wellness recommendation related to the received email to be surfaced.

11. The system of claim 10, wherein the plurality of signals further comprises at least one of:
a number of times the received email was scrolled through; and
a number of highlights made to the received email.

12. The system of claim 10, wherein the plurality of signals further comprises a natural language input included in the email.

13. The system of claim 12, wherein the one or more processors are further responsive to the computer-executable instructions contained in the program code and operative to:
apply a natural language processing model to the natural language input, wherein the natural language processing model has been trained to classify natural language inputs into tone categories.

14. The system of claim 10, wherein the threshold baseline value is a baseline for emails the user receives from an email account that the received email was sent from.

15. The system of claim 10, wherein the biometric signal associated with the user further comprises:
an image of facial features of the user during a time that the received email was being reviewed by the user.

16. The system of claim 15, wherein the one or more processors are further responsive to the computer-executable instructions contained in the program code and operative to:
apply a machine learning model to the image, wherein the machine learning model has been trained to classify facial feature images into expression type categories.

17. The system of claim 10, wherein the biometric signal associated with the user further comprises:
an audio recording of the user's voice taken during a time that the received email was open in an email application associated with the user.

18. The system of claim 10, wherein in generating the anxiety score associated with the received email the one or more processors are further responsive to the computer-executable instructions contained in the program code and operative to:
analyze a plurality of lexical features included in the audio recording; and
analyze a plurality of prosodic features included in the audio recording.

19. A computer-readable storage device comprising executable instructions that, when executed by one or more processors, assist with surfacing wellness recommendations, the computer-readable storage device including instructions executable by the one or more processors for:
receiving a plurality of signals related to a user, the plurality of signals comprising:
an active duration of time spent reviewing a received email, and
a biometric signal associated with the user comprising at least one of:
a blood pressure value for the user during a time that the received email was open in an email application associated with the user, and
a heartrate value for the user during a time that the received email was open in an email application associated with the user;
generating an anxiety score associated with the received email;
determining that the anxiety score is above a threshold baseline value for the user; and
causing a wellness recommendation related to the received email to be surfaced.

20. The computer-readable storage device of claim 19, wherein the threshold baseline value is a baseline for emails the user receives from an email account that the received email was sent from.
